(19) United States
(12) Patent Application Publication
(10) Pub. No.: US 2020/0009156 A1
(43) Pub. Date: Jan. 9, 2020

(54) FLUTICASONE EXTENDED-RELEASE FORMULATIONS AND METHODS OF USE THEREOF

(71) Applicant: Flexion Therapeutics, Inc., Burlington, MA (US)

(72) Inventors: Neil BODICK, Burlington, MA (US); John Derek JACKSON, Burlington, MA (US); Ujjwal JOSHI, Burlington, MA (US)

(21) Appl. No.: 16/493,437

(22) PCT Filed: Mar. 16, 2018
(86) PCT No.: PCT/US2018/022848

§ 371 Date: Sep. 12, 2019

Related U.S. Application Data
(60) Provisional application No. 62/473,029, filed on Mar. 17, 2017.

(51) Int. Cl. A61K 31/56; A61K 9/16; A61K 9/00

(52) U.S. Cl. CPC A61K 31/56; A61K 9/0019; A61K 9/1647

(57) ABSTRACT

This disclosure relates to extended-release fluticasone propionate (FP) formulations that include small microparticles and/or nanoparticles and methods of using these extended-release FP formulations for treating pain and/or inflammation in a subject.

### RELATED APPLICATIONS

This application claims priority to U.S. Application No. 62/473,029, filed Mar. 17, 2017, the contents of which are herein incorporated by reference in their entirety.

### FIELD OF THE INVENTION

This disclosure relates to extended-release fluticasone propionate (FP) formulations that include small microparticles and/or nanoparticles and methods of using these extended-release FP formulations for treating pain and/or inflammation in a subject.

### BACKGROUND OF THE INVENTION

Corticosteroids influence all tissues of the body and produce various cellular effects. These steroids regulate carbohydrate, lipid, protein biosynthesis and metabolism, and water and electrolyte balance. Corticosteroids influencing cellular biosynthesis or metabolism are referred to as glucocorticoids while those affecting water and electrolyte balance are mineralocorticoids. Both glucocorticoids and mineralocorticoids are released from the cortex of the adrenal gland.

Corticosteroids are used in the treatment of a variety of indications, including immediate release formulations for use in treating disorders and diseases associated with joint pain and/or inflammation. Steroid injections for lower back pain may provide some relief for certain patients, but any benefits are temporary.

Accordingly, there is a medical need for therapies for lower back pain and/or inflammation that provide maximal analgesic effect over a prolonged duration and minimize undesirable side effects.

### SUMMARY OF THE INVENTION

Disclosed herein are extended-release formulations comprising nanoparticles, small microparticles or a combination thereof, wherein the small microparticles and/or nanoparticles comprise fluticasone propionate (FP) or a commercially available chemical analogue or a pharmaceutically-acceptable salt thereof and a polymer matrix. Also disclosed herein are extended-release formulations comprising nanoparticles, small microparticles, or a combination thereof, wherein the small microparticles and/or nanoparticles comprise fluticasone propionate (FP) or a pharmaceutically-acceptable salt thereof and a polymer matrix. In some embodiments, the polymer matrix comprises a poly(lactic-co-glycolic) acid copolymer (PLGA) copolymer matrix. In some embodiments, the polymer matrix is a poly(lactic-co-glycolic) acid copolymer (PLGA) copolymer matrix. In some embodiments, the polymer matrix comprises a polylactic acid (PLA) matrix. In some embodiments, the polymer matrix is a polylactic acid (PLA) matrix. Also disclosed herein are methods of using such FP extended-release formulations, also referred to herein as FP/polymer nanoparticles, FP/polymer small microparticles, FP/polymer formulations, FP/PLGA nanoparticles, FP/PLGA small microparticles, FP/PLGA formulations, FP/PLA nanoparticles, FP/PLA small microparticles, FP/PLA formulations, and all variations thereof, in the treatment of pain and/or inflammation in a subject.

In some embodiments, the FP is released for at least greater than 14 days. In some embodiments, the FP is released for between 14 days and 90 days. In some embodiments, the FP is released for between 14 days and 180 days. In some embodiments, the extended release formulation is a controlled- or sustained-release formulation.

The extended release FP formulations provided herein are useful in the treatment of a variety of pain and/or inflammation indications in a subject. Also disclosed herein, therefore, are methods of treating pain and/or inflammation in a patient comprising administering to said patient a therapeutically effective amount of an extended-release formulation as described herein. Also disclosed herein is an extended-release formulation as described herein for use in treating pain and/or inflammation. In some embodiments, the pain and/or inflammation is pain and/or inflammation in an epidural space. In some embodiments, the pain and/or inflammation is back pain and/or inflammation. In some embodiments, the pain and/or inflammation is lower back pain and/or inflammation. In some embodiments, the pain and/or inflammation is pain and/or inflammation in a joint. In some embodiments, the pain and/or inflammation is pain and/or inflammation in a synovial joint. In some embodiments, the pain and/or inflammation is pain and/or inflammation in a small to medium synovial joint. In some embodiments, the pain and/or inflammation is pain and/or inflammation in a large synovial joint. In some embodiments, the pain and/or inflammation is caused by, related to, or otherwise associated with an ophthalmic indication (ophthalmic pain and/or inflammation). In some embodiments, the pain and/or inflammation is a side effect of an injection in the subject. In some embodiments, the pain and/or inflammation is an acute injury or a side effect thereof. In some embodiments, the pain and/or inflammation is a chronic injury or a side effect thereof. In some embodiments, the pain and/or inflammation is a chronic disorder. In some embodiments, the pain and/or inflammation is a side effect of surgical intervention in the subject. In some embodiments, the pain and/or inflammation is related to a partial or total joint replacement or a side effect thereof.

In some embodiments, the compositions are extended release formulations in which the fluticasone propionate is released over a prolonged period of time. In some embodiments, the compositions are extended release formulations in which the FP is released for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, at least 24 weeks or more, at least 25 weeks or more, at least 26 weeks or more at least 27 weeks or more, at least 28 weeks or more, at least 29 weeks or more, at least 30 weeks or more, at least 31 weeks or more, at least 32 weeks or more, at least 33 weeks or more, at least 34 weeks or more, at least 35 weeks or more, and/or at least 36 weeks or more.

In some embodiments, the extended release formulations are sustained- or controlled-release formulations in which the fluticasone propionate is released at a uniform or substantially uniform rate over a prolonged period of time. In some embodiments, the extended release formulations are sustained- or controlled-release formulations in which the FP is released at a uniform or substantially uniform rate for at least four weeks or more, at least five weeks or more, at least six weeks or more, at least seven weeks or more, at least eight weeks or more, at least nine weeks or more, at least 10 weeks or more, at least 11 weeks or more, at least 12 weeks or more, at least 13 weeks or more, at least 14 weeks or more, at least 15 weeks or more, at least 16 weeks or more, at least 17 weeks or more, at least 18 weeks or more, at least 19 weeks or more, at least 20 weeks or more, at least 21 weeks or more, at least 22 weeks or more, at least 23 weeks or more, at least 24 weeks or more, at least 25 weeks or more, at least 26 weeks or more at least 27 weeks or more, at least 28 weeks or more, at least 29 weeks or more, at least 30 weeks or more, at least 31 weeks or more, at least 32 weeks or more, at least 33 weeks or more, at least 34 weeks or more, at least 35 weeks or more, and/or at least 36 weeks or more.

In some embodiments, the small microparticles have a mean diameter of between 1 μm to 10 μm, for example, the small microparticles have a mean diameter in the range of 1-10 μm, 1-9 μm, 1-8 μm, 1-7 μm, 1-6, μm, 1-5 μm, 1-4 μm, 1-3 μm, 2-9 μm, 2-8 μm, 2-7 μm, 2-6 μm, 2-5 μm, 2-4 μm, 2-3 μm, 3-9 μm, 3-8 μm, 3-7 μm, 3-6 μm, 3-5 μm, 3-4 μm, 4-9 μm, 4-8 μm, 4-7 μm, 4-6 μm, 4-5 μm, 5-9 μm, 5-8 μm, 5-7 μm, 5-6 μm, 6-9 μm, 6-8 μm, 6-7 μm, 7-9 μm, 7-8 μm, or 8-9 μm. In some embodiments, the small microparticles have a mean diameter of between 1 μm to 3 μm. In some embodiments, the small microparticles have a mean diameter of between 1 μm to 6 μm. In some embodiments, the small microparticles have a mean diameter of between 3 μm to 6 μm. In some embodiments, the small microparticles have a mean diameter of about 10 μm. In some embodiments, the small microparticles have a mean diameter of about 6 μm. In some embodiments, the small microparticles have a mean diameter of about 1 μm. It is understood that these ranges refer to the mean diameter of all small microparticles in a given population. The diameter of any given individual small microparticles could be within a standard deviation above or below the mean diameter.

In some embodiments, the nanoparticles have a mean diameter of between 100 nm to 1000 nm, for example, the nanoparticles have a mean diameter in the range of 200 nm to 1000 nm, 300 nm to 1000 nm, 400 nm to 1000 nm, 500 nm to 1000 nm, 600 nm to 1000 nm, 700 nm to 1000 nm, 800 nm to 1000 nm, 900 nm to 1000 nm, 100 nm to 900 nm, 200 nm to 900 nm, 300 nm to 900 nm, 400 nm to 900 nm, 500 nm to 900 nm, 600 nm to 900 nm, 700 nm to 900 nm, 800 nm to 900 nm, 100 nm to 800 nm, 200 nm to 800 nm, 300 nm to 800 nm, 400 nm to 800 nm, 500 nm to 800 nm, 600 nm to 800 nm, 700 nm to 800 nm, 100 nm to 700 nm, 200 nm to 700 nm, 300 nm to 700 nm, 400 nm to 700 nm, 500 nm to 700 nm, 600 nm to 700 nm, 100 nm to 600 nm, 200 nm to 600 nm, 300 nm to 600 nm, 400 nm to 600 nm, 500 nm to 600 nm, 100 nm to 500 nm, 200 nm to 500 nm, 300 nm to 500 nm, 400 nm to 500 nm, 100 nm to 400 nm, 200 nm to 400 nm, 300 nm to 400 nm, 100 nm to 300 nm, 200 nm to 300 nm, 100 nm to 200 nm, 110 nm to 200, 120 nm to 200 nm, 130 nm to 200 nm, 140 nm to 200 nm, 150 nm to 200 nm, 160 nm to 200 nm, 170 nm to 200 nm, 180 nm to 200 nm, or 190 nm to 200 nm. In some embodiments, the nanoparticles have a mean diameter of between 100 nm to 500 nm. In some embodiments, the nanoparticles have a mean diameter of between 120 nm to 500 nm. In some embodiments, the nanoparticles have a mean diameter of between 150 nm to 500 nm. In some embodiments, the nanoparticles have a mean diameter of between 250 nm to 500 nm. In some embodiments, the nanoparticles have a mean diameter of between 100 nm to 250 nm. In some embodiments, the nanoparticles have a mean diameter of between 120 nm to 250 nm. In some embodiments, the nanoparticles have a mean diameter of between 150 nm to 250 nm. In some embodiments, the nanoparticles have a mean diameter of between 200 nm to 250 nm. In some embodiments, the nanoparticles have a mean diameter of between 100 nm to 150 nm. In some embodiments, the nanoparticles have a mean diameter of between 100 nm to 120 nm. In some embodiments, the nanoparticles have a mean diameter of about 500 nm. In some embodiments, the nanoparticles have a mean diameter of about 250 nm. In some embodiments, the nanoparticles have a mean diameter of about 150 nm. In some embodiments, the nanoparticles have a mean diameter of about 120 nm. In some embodiments, the nanoparticles have a mean diameter of about 100 nm. It is understood that these ranges refer to the mean diameter of all nanoparticles in a given population. The diameter of any given individual nanoparticle could be within a standard deviation above or below the mean diameter.

In some embodiments, the formulations include a combination of small microparticles and nanoparticles. In some embodiments, the small microparticles in the combination have a mean diameter of between 1 μm to 10 μm, for example, the small microparticles have a mean diameter in the ranges described above. In some embodiments, the small microparticles in the combination have a mean diameter of between 1 μm to 3 μm. In some embodiments, the small microparticles in the combination have a mean diameter in a range of between 1 μm to 6 μm. In some embodiments, the small microparticles in the combination have a mean diameter of between 3 μm to 6 μm. In some embodiments, the small microparticles in the combination have a mean diameter selected from the group consisting of about 10 μm, about 6 μm, and about 1 μm. In some embodiments, the nanoparticles in the combination have a mean diameter of between 100 nm to 1000 nm, for example, the nanoparticles in the combination have a mean diameter in the ranges described above. In some embodiments, the nanoparticles in the combination have a mean diameter in a range selected from the group consisting of between 100 nm to 500 nm, between 120 nm to 500 nm, between 150 nm to 500 nm, between 250 nm to 500 nm, between 100 nm to 250 nm, between 120 nm to 250 nm, between 150 nm to 250 nm, between 200 nm to 250 nm, between 100 nm to 150 nm, and between 100 nm to 120 nm. In some embodiments, the nanoparticles in the combination have a mean diameter selected from the group consisting of about 500 nm, about 250 nm, about 150 nm about 120 nm, and about 100 nm. It is understood that these ranges refer to the mean diameter of all small microparticles and/or nanoparticles in a given population. The diameter of any given individual small microparticles and/or nanoparticles could be within a standard deviation above or below the mean diameter. In some embodiments, the small microparticles in the combination have a mean diameter of between 1 μm to 10 μm and the nanoparticles in the combination have a mean diameter of between 100 nm to 1000 nm, between 100 nm to 500 nm, between 120 nm to 500 nm, between 150 nm to 500 nm, between 250 nm to 500 nm, between 100 nm to 250 nm, between 120 nm to 250 nm, between 150 nm to 250 nm, between 200 nm to 250 nm, between 100 nm to 150 nm, or between 100 nm to 120 nm. In some embodiments, the small microparticles in the combination have a mean diameter in a range of between 1 μm to 6 μm and the nanoparticles in the combination have a mean diameter of between 100 nm to 1000 nm, between 100 nm to 500 nm, between 120 nm to 500 nm, between 150 nm to 500 nm, between 250 nm to 500 nm, between 100 nm to 250 nm, between 120 nm to 250 nm, between 150 nm to 250 nm, between 200 nm to 250 nm, between 100 nm to 150 nm, or between 100 nm to 120 nm. In some embodiments, the small microparticles in the combination have a mean diameter of between 1 μm to 3 μm and the nanoparticles in the combination have a mean diameter of between 100 nm to 1000 nm, between 100 nm to 500 nm, between 120 nm to 500 nm, between 150 nm to 500 nm, between 250 nm to 500 nm, between 100 nm to 250 nm, between 120 nm to 250 nm, between 150 nm to 250 nm, between 200 nm to 250 nm, between 100 nm to 150 nm, or between 100 nm to 120 nm. In some embodiments, the small microparticles in the combination have a mean diameter of between 3 μm to 6 μm and the nanoparticles in the combination have a mean diameter of between 100 nm to 1000 nm, between 100 nm to 500 nm, between 120 nm to 500 nm, between 150 nm to 500 nm, between 250 nm to 500 nm, between 100 nm to 250 nm, between 120 nm to 250 nm, between 150 nm to 250 nm, between 200 nm to 250 nm, between 100 nm to 150 nm, or between 100 nm to 120 nm.

In some embodiments, the size range of the particles used in the FP/polymer extended-release formulations provided herein is tailored or otherwise modified based on the desired indication that is to be treated. For example, particles having a mean diameter larger than 6-7 μm are at risk for adverse events when administered to the spine, such as, for example, arterial infarction in the spine. However, to ensure maximal release of the FP from the FP/polymer formulation, it is desirable to maximize particle size, while simultaneously constraining the particle size distribution to exclude the risk of individual particles or aggregates above 6-7 μm. Accordingly, in some embodiments for the treatment of spinal-related indications, the particles used in the one or more epidural injections in these embodiments can be in the 100 nm to 6 μm range, provided that the upper end of this range is subject to stringent particle control and distribution conditions and techniques. In some embodiments for the treatment of spinal-related indications, the particles used in the one or more epidural injections is in the range of about 100 nm to 3 μm range.

In some embodiments for the treatment of synovial joint-related indications, the particles used in the one or more injections in these embodiments can be in the 100 nm to 10 μm range, as the use of larger diameter particles can maximize joint residency time and/or can maximize drug release into the synovial joint cavity and surrounding areas, while minimizing the available surface area of the particle. In some embodiments for the treatment of synovial joint-related indications, the particles used in the one or more injections in these embodiments are in the 100 nm to 6 μm range. In some embodiments for the treatment of synovial joint-related indications, the particles used in the one or more injections in these embodiments are in the 100 nm to 3 μm range.

In some embodiments, the small microparticles and/or nanoparticles further comprise a polyethylene glycol (PEG) moiety, wherein the PEG moiety comprises between 25% to 0% weight percent of the small microparticles and/or nanoparticle. (As used herein, the expression “wherein the PEG moiety comprises between 25% to 0% weight percent of the small microparticles and/or nanoparticle” is understood to mean that the small microparticles and/or nanoparticles comprise between 25% to 0% of the PEG moiety, by weight of the small microparticles and/or nanoparticles). In some embodiments, the PEG moiety is present as a PEG/PLGA block co-polymer. In some embodiments, the PEG moiety is included in the extended-release formulation as a separate excipient. In some embodiments of the small microparticles and/or nanoparticles that include a PEG moiety, the populations, preparations and/or formulations of the invention do not require the presence of PEG to exhibit the desired FP extended and/or sustained release kinetics and bioavailability profile.

FP formulations of the disclosure produce and maintain a local concentration of FP at or near the injection site, for example, the epidural or synovial fluid concentration of FP, that produces maximal analgesic effect for an extended period of time, e.g., at least 24 days, at least 42 days, at least 49 days, at least 50 days, at least 55 days, at least 56 days, at least 60 days, at least 63 days, at least 65 days, at least 70 days, at least 75 days, at least 77 days, at least 80 days, at least 84 days, at least 85 days, at least 90 days, at least four months or longer, at least five months or longer, at least six months or longer, at least seven months or longer, at least eight months or longer, at least nine months or longer, at least 10 months or longer, at least 11 months or longer, or at least 12 months or longer. As used herein, the term “maximal analgesic effect” and variations thereof is a level of analgesic effect, observed after administration of a formulation of the disclosure, which is greater than the acute analgesic effect provided by standard, non-extended release FP suspensions.

Ranges of local FP concentrations at or near the injection site, e.g., epidural or synovial concentrations, are provided throughout the disclosure. Those of ordinary skill in the art will appreciate that these ranges are values based on samples from various subjects, e.g., the mean value from the detected levels at which the patients reported maximal analgesic effect. These values may vary slightly from sample to sample. Thus, the ranges of local FP concentrations, e.g., epidural or synovial concentrations, provided herein are a target local concentration, e.g., a target epidural or synovial concentration, and patients having local FP concentrations, e.g., epidural or synovial concentrations, slightly outside the ranges provided herein, e.g., between about 0.95 ng/ml to about 157.58 ng/ml for fluticasone propionate, may still achieve and maintain maximal analgesic effect over a prolonged duration.

In some embodiments, the local FP concentrations at or near the site of injection, e.g., epidural or synovial concentration of fluticasone propionate is maintained at a level in the range of about 0.95 ng/ml to about 157.58 ng/ml, or any value in between the range of about 0.95 ng/ml to about 157.58 ng/ml.

In some embodiments, the local concentration of the fluticasone propionate, e.g., the epidural or synovial concentration of the fluticasone propionate, is maintained by administering at least one additional dose of FP. In some embodiments, at least one additional dose of FP is administered as an extended release, e.g., a controlled- or sustained-release formulation. In some embodiments, FP is released from the formulation for a duration of at least between 14 days and 90 days. In some embodiments, FP is released from the formulation for a duration of at least between 30 days and 90 days. In some embodiments, FP is released from the formulation for a duration of at least 3 months. In some embodiments, FP is released from the formulation for a duration of at least between 3 months and 12 months. In some embodiments, FP is released from the formulation for a duration of at least between 3 months and 6 months. In some embodiments, FP is released from the formulation for a duration of at least between 6 months and 12 months.

In some embodiments, the fluticasone propionate is released from the formulation for a duration of at least between 14 days and 90 days. In some embodiments, FP is released from the formulation for a duration of at least between 30 days and 90 days. In some embodiments, FP is released from the formulation for a duration of at least 3 months. In some embodiments, FP is released from the formulation for a duration of at least between 3 months and 12 months. In some embodiments, FP is released from the formulation for a duration of at least between 3 months and 6 months. In some embodiments, FP is released from the formulation for a duration of at least between 6 months and 12 months.

In some embodiments, the formulation is administered as one or more injections. In some embodiments, the injection is one or more local injections at a site of pain. In some embodiments, the injection is one or more epidural injections. In some embodiments, the injection is one or more synovial injections. In some embodiments, the injection is one or more intra-articular injections. In some embodiments, the injection is one or more intrathecal injections. In some embodiments, the injection is one or more intra-bursal injections.

In some embodiments, the patient has chronic lower back pain and/or inflammation. In some embodiments, the patient has chronic lower back pain. In some embodiments, the patient has acute lower back pain and/or inflammation. In some embodiments, the patient has acute lower back pain. In some embodiments, the patient has radiculopathy due to disc herniation.

In some embodiments, the patient has pain and/or inflammation in a small to medium synovial joint. Examples of small to medium synovial joints include, by way of non-limiting example, interphalangeal joints, metacarpophalangeal joints, elbow joints, wrist joints, and/or ankle joints. In some embodiments, the pain and/or inflammation in the small to medium synovial joint is caused by inflammatory diseases such as osteoarthritis, rheumatoid arthritis, acute gouty arthritis, or synovitis. In some embodiments, the patient with pain and/or inflammation in a small to medium synovial joint has osteoarthritis.

In some embodiments, the patient has pain and/or inflammation in a large synovial joint. Examples of large synovial joints include, by way of non-limiting example, knee joints, shoulder joints, and/or hip joints. In some embodiments, the large synovial joint is one or more knee joints. In some embodiments, the pain and/or inflammation in the large synovial joint is caused by inflammatory diseases such as osteoarthritis, rheumatoid arthritis, acute gouty arthritis, or synovitis. In some embodiments, the patient with pain and/or inflammation in a large synovial joint (for example, one or more knee joints) has osteoarthritis.

In some embodiments, the patient has pain and/or inflammation related to another indication or disorder associated with pain and/or inflammation. In some embodiments, the patient has carpel tunnel syndrome. In some embodiments, the patient is suffering one or more side effects from a trigger point injection. In some embodiments, the patient is suffering from one or more side effects of an injection in a joint such as, for example, an injection in one or more of the following joints: knee, shoulders, wrists, fingers, ankles, or any combination of these joints. In some embodiments, the patient has rheumatoid arthritis. In some embodiments, the patient is suffering from one or more side effects of an injection in a joint for the treatment of rheumatoid arthritis such as, for example, an injection in one or more of the following joints: knee, shoulders, wrists, fingers, ankles, or any combination of these joints. In some embodiments, the patient has cervical radiculopathy. In some embodiments, the patient is suffering one or more side effects from surgical intervention in the subject. In some embodiments, the patient is suffering one or more side effects from a partial or total joint replacement, such as, for example, a knee or hip replacement. In some embodiments, the patient has Complex Regional Pain Syndromes (CRPS). In some embodiments, the patient has lumbar spinal stenosis. In some embodiments, the patient has an acute spinal injury. In some embodiments, the patient has discogenic pain.

In some embodiments, the patient has pain and/or inflammation caused by or otherwise associated with an ophthalmic indication. In some embodiments, the pain and/or inflammation caused by or otherwise associated with an ophthalmic indication is selected from the group consisting of endophthalmitis (e.g., the endogenous form and the exogenous form), macular edema (e.g., macular edema that occurs as a result of age-related macular degeneration, cataract surgery, diabetes, drug toxicity, eye injury, retinal vein occlusion (e.g., central retinal vein occlusion (CRVO) and branch retinal vein occlusion), or other inflammatory eye diseases, e.g., pseudophakic macular edema), conjunctivitis, diabetic retinopathy, dry eye, episcleritis, keratitis, optic neuritis, orbital inflammation, orbital pseudotumor, post-operative inflammation, proliferative vitreoretinopathy, retinal vasculitis, scleritis, and uveitis (e.g., (i) uveitis associated with sepsis (e.g., lipopolysaccharide (LPS)-induced uveitis); (ii) autoimmune uveitis (e.g., uveitis associated with lupus); or (iii) uveitis associated with type II, type III, type IV, or type V hypersensitivity reactions).

The FP formulations provided herein are effective at treating pain and/or inflammation with minimal long-term side effects associated with corticosteroid administration. In some embodiments, the FP formulations provided herein maintain sufficiently high local concentrations of FP at or near the site of injection to be effective at treating pain and/or inflammation while producing sufficiently low systemic concentrations of FP to avoid adverse suppression of the hypothalamic-pituitary-adrenal (HPA) axis. In some embodiments, the FP formulations provided herein maintain sufficiently high epidural concentrations of FP to be effective at treating pain and/or inflammation while producing sufficiently low systemic concentrations of FP to avoid adverse suppression of the HPA axis. In some embodiments, the FP formulations provided herein maintain sufficiently high synovial concentrations of FP to be effective at treating pain and/or inflammation while producing sufficiently low systemic concentrations of FP to avoid adverse suppression of the HPA axis.

The FP formulations are suitable for administration, for example, local administration by injection into a site at or near the site of a patient's pain and/or inflammation. In some embodiments, an extended release formulation of FP and a PLGA copolymer matrix is administered locally to treat pain and inflammation. Local administration of an FP formulation can occur, for example, by injection into the epidural space at or near the site of a patient's pain. Local administration of an FP formulation can occur, for example, by injection into the synovial joint cavity at or near the site of a patient's pain. Local administration of the FP formulation can occur, for example, by epidural, intra-articular, intrathecal, or intra-bursal administration.

In some embodiments, an extended release form, e.g., a sustained release form, of FP is administered (e.g., by single injection or as sequential injections) into an epidural space for the treatment of lower back pain. In some embodiments, an extended release form such as, e.g., a sustained release form, of FP is administered (e.g., by single injection or as sequential injections) into an epidural space to slow, arrest, reverse or otherwise inhibit structural damage to tissues associated with lower back pain. Because both pain and structural progression are the product of local inflammation in some lower back pain disorders, and because FP acts through the reduction of inflammation, the concentration ranges cited here for maximizing analgesic effect will also be effective in slowing or stopping structural progression. In some embodiments, an extended release form, e.g., a sustained release form, of FP is administered (e.g., by single injection or as sequential injections) into a synovial joint cavity for the treatment of pain and/or inflammation in a small to medium synovial joint. In some embodiments, an extended release form such as, e.g., a sustained release form, of FP is administered (e.g., by single injection or as sequential injections) into a synovial joint cavity to slow, arrest, reverse or otherwise inhibit structural damage to tissues associated with pain and/or inflammation in a small to medium synovial joint. Because both pain and structural progression are the product of local inflammation in some pain disorders in small to medium synovial joints, and because FP acts through the reduction of inflammation, the concentration ranges cited here for maximizing analgesic effect will also be effective in slowing or stopping structural progression in small to medium synovial joints.

In some embodiments, an extended release form, e.g., a sustained release form, of FP is administered (e.g., by single injection or as sequential injections) into a synovial joint cavity for the treatment of pain and/or inflammation in a large synovial joint. In some embodiments, an extended release form such as, e.g., a sustained release form, of FP is administered (e.g., by single injection or as sequential injections) into a synovial joint cavity to slow, arrest, reverse or otherwise inhibit structural damage to tissues associated with pain and/or inflammation in a large synovial joint. Because both pain and structural progression are the product of local inflammation in some pain disorders in large synovial joints, and because FP acts through the reduction of inflammation, the concentration ranges cited here for maximizing analgesic effect will also be effective in slowing or stopping structural progression in large synovial joints. The FP formulations described herein are also useful in the treatment of a systemic disorder for which FP treatment would be required or otherwise therapeutically beneficial.

These FP formulations, when administered to a patient, exhibit an improved benefit or other therapeutic outcome in the treatment of a disease, for example a lower back pain related disorder, pain and/or inflammation in a small to medium synovial joint, or pain and/or inflammation in a large synovial joint, as compared to the administration, for example administration into the epidural space or into the synovial joint cavity, of an equivalent amount of a non-extended release FP formulation or suspension, absent any particulate suspension, small microparticle, nanoparticle, or other type of extended-release formulation, incorporation, admixture, or encapsulation. The improved benefit can be any of a variety of laboratory or clinical results. For example, administration of an extended-release FP formulation is considered more successful than administration of FP absent any small microparticle, nanoparticle or other extended-release formulation if, following administration of the extended-release FP formulations, one or more of the symptoms associated with the disease is alleviated, reduced, inhibited or does not progress to a further, i.e., worse, state, to a greater extent than the level that is observed after administration of FP absent any small microparticle, nanoparticle or other extended-release formulation. Administration of an extended-release FP formulation is considered more successful than administration of FP absent any small microparticle, nanoparticle or other extended-release formulation if, following administration of the extended-release FP formulations, anti-inflammatory activity is sustained for a longer period than the level that is observed after administration of FP absent any small microparticle, nanoparticle and/or any other extended-release formulation.

The FP formulations provided herein can be used in combination with any of a variety of therapeutics, also referred to herein as “co-therapies.”

For example, the FP formulations can be used in combination with a non-extended release FP (or other corticosteroid) solution or suspension, which provides high local exposures for between 1 day and 14 days following administration and which produce systemic exposures that may be associated with transient suppression of the HPA axis. In some embodiments, the same corticosteroid, i.e., FP, is used in both the non-extended release component and sustained release components. In some embodiments, the non-extended release component contains a corticosteroid that is different from that of the extended release FP formulation, i.e., the non-extended release component does not include FP.

In some co-therapy embodiments, the period of extended release is between 30 days and 12 months. In some co-therapy embodiments, the period of extended release is between 90 days and 12 months. In some co-therapy embodiments, the period of extended release is at least 3 months. In some co-therapy embodiments, the period of extended release is at least between 3 months and 12 months. In some co-therapy embodiments, the period of extended release is at least between 3 months and 6 months. In some co-therapy embodiments, the period of extended release is at least between 6 months and 12 months. In some co-therapy embodiments, the period of sustained release is between 30 days and 12 months. In some co-therapy embodiments, the period of sustained release is between 90 days and 12 months. In some co-therapy embodiments, the period of sustained release is at least 3 months. In some co-therapy embodiments, the period of sustained release is at least between 3 months and 12 months. In some co-therapy embodiments, the period of sustained release is at least between 3 months and 6 months. In some co-therapy embodiments, the period of sustained release is at least between 6 months and 12 months.

In some co-therapy embodiments, the high local exposure attributable to the non-extended release component lasts for between 1 day and 28 days. In some co-therapy embodiments, the high local exposure attributable to the non-extended release component lasts for between 1 day and 21 days. In some co-therapy embodiments, the high local exposure attributable to the non-extended release component lasts for between 1 day and 14 days. In some co-therapy embodiments, the high local exposure attributable to the non-extended release component lasts for between 1 day and 10 days. In some co-therapy embodiments, the high local exposure attributable to the non-extended release component lasts between 1 days and 8 days. In some co-therapy embodiments, the high local exposure attributable to the non-extended release component lasts between 1 days and 6 days. In some co-therapy embodiments, the high local exposure attributable to the non-extended release component lasts for between 1 day and 4 days.

Suitable additional agents for use in combination with the FP formulations provided herein include hyaluronic acid preparations including but not limited to Synvisc One, Gel 200 and Supartz; nonsteroidal anti-inflammatory drugs (NSAIDS) including but not limited to aspirin, celecoxib (Celebrex), diclofenac (Voltaren), diflunisal (Dolobid), etodolac (Lodine), ibuprofen (Motrin), indomethacin (Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin); biologics including but not limited to Actemra (tocilizumab), Enbrel (etanercept), Humira (adalimumab), Kineret (anakinra), Orencia (abatacept), Remicade (infliximab), Rituxan (rituximab), Cimzia (certolizumab), and Simponi (golimumab); disease modifying agents including but not limited to methotrexate, Plaquenil (hydroxychloroquine) and Azulfidine (sulfasalazine), Minocin (minocycline); and other analgesic and anti-inflammatory agents including but not limited to p38 inhibitors, Janus kinase (JAK) inhibitors, opioids, other corticosteroids, lidocaine, bupivacaine, ropivacaine, botulinum toxin A.

In some co-therapy embodiments, the FP formulation and additional agent are formulated into a single therapeutic composition, and the FP formulation and additional agent are administered simultaneously. Alternatively, the FP formulation and additional agent are separate from each other, e.g., each is formulated into a separate therapeutic composition, and the FP formulation and the additional agent are administered simultaneously, or the FP formulation and the additional agent are administered at different times during a treatment regimen. For example, the FP formulation is administered prior to the administration of the additional agent, the FP formulation is administered subsequent to the administration of the additional agent, or the FP formulation and the additional agent are administered in an alternating fashion. As described herein, the FP formulation and additional agent are administered in single doses or in multiple doses.

In some embodiments, the FP formulation and the additional agent are administered by the same route. In some embodiments, the FP formulation and the additional agent are administered via different routes.

It is contemplated that whenever appropriate, any embodiment of the present disclosure can be combined with one or more other embodiments of the present disclosure, even though the embodiments are described under different aspects of the present disclosure.

### BRIEF DESCRIPTION OF THE FIGURES

FIG. 1 is a graph depicting the in vitro release profile of FP formulations of the disclosure.

FIG. 2 is a graph showing the in vitro release profile of FP nano-formulations of the disclosure.

FIG. 3 is a schematic detailing the single emulsion nano-particle fabrication process.

### DETAILED DESCRIPTION OF THE INVENTION

The disclosure provides extended release formulations and methods for the treatment of pain and/or inflammation, including lower back pain and/or inflammation or pain, inflammation in a small to medium synovial joint, or pain and/or inflammation in a large synovial joint, using fluticasone propionate or a commercially available chemical analogue or a pharmaceutically acceptable salt thereof. The compositions and methods provided herein use extended release formulations that include fluticasone propionate or a commercially available chemical analogue or a pharmaceutically acceptable salt thereof and a poly(lactic-co-glycolic) acid copolymer (PLGA) copolymer matrix.

The extended release FP formulations provided herein are useful in the treatment of a variety of pain and/or inflammation indications in a subject. In some embodiments, the pain and/or inflammation is pain and/or inflammation in an epidural space. In some embodiments, the pain and/or inflammation is lower back pain and/or inflammation. In some embodiments, the pain and/or inflammation is pain and/or inflammation in a joint. In some embodiments, the pain and/or inflammation is pain and/or inflammation in a synovial joint. In some embodiments, the pain and/or inflammation is pain and/or inflammation in a small to medium synovial joint. In some embodiments, the pain and/or inflammation is pain and/or inflammation in a large synovial joint. In some embodiments, the pain and/or inflammation is caused by, related to, or otherwise associated with an ophthalmic indication. In some embodiments, the pain and/or inflammation is a side effect of an injection in the subject. In some embodiments, the pain and/or inflammation is an acute injury or a side effect thereof. In some embodiments, the pain and/or inflammation is a chronic injury or a side effect thereof. In some embodiments, the pain and/or inflammation is a chronic disorder. In some embodiments, the pain and/or inflammation is a side effect of surgical intervention in the subject. In some embodiments, the pain and/or inflammation is related to a partial or total joint replacement or a side effect thereof.

The FP/polymer extended-release formulations provided herein are effective at treating lower back pain and/or inflammation with minimal long term side effects commonly seen with corticosteroid administration. These compositions and methods are useful in patients with lower back pain and other diseases and disorders associated with pain and/or inflammation in the lower back. These compositions and methods are useful in patients with pain and/or inflammation in a small to medium synovial joint, such as, by way of non-limiting example, one or more interphalangeal joints, one or more metacarpophalangeal joints, one or more an elbow joints, one or more wrist joints, one or more ankle joints, and any combination thereof. In some embodiments, the patient with pain and/or inflammation in a small to medium synovial joint has osteoarthritis. These compositions and methods are useful in patients with pain and/or inflammation in a large synovial joint, such as, by way of non-limiting example, one or more knee joints, one or more shoulder joints, one or more hip joints, and any combination thereof. In some embodiments, the patient with pain and/or inflammation in a large synovial joint has osteoarthritis.

In some embodiments, an extended release formulation of fluticasone propionate and polymer matrix is administered locally to treat lower back pain and inflammation. In some embodiments, the lower back pain is radiculopathy due to lumbar disc herniation. Local administration of an FP/polymer extended-release formulation can occur, for example, by injection into the epidural space, intra-articular space, peri-articular space, soft tissues, lesions, perineural space, or the foramenal space at or near the site of a patient's pain. In some embodiments, an extended release form of FP is administered (e.g., by single injection or as sequential injections) into an epidural space for the treatment of pain, for example, due to lower back pain or other lower back disorder. In some embodiments, an extended release formulation of FP and a PLGA copolymer matrix is administered (e.g., by single injection or as sequential injections) into an epidural space or into soft tissues to slow, arrest, reverse or otherwise inhibit structural damage to tissues associated with lower back pain. In some embodiments, an extended release formulation of FP and a PLA copolymer matrix is administered (e.g., by single injection or as sequential injections) into an epidural space or into soft tissues to slow, arrest, reverse or otherwise inhibit structural damage to tissues associated with lower back pain.

The formulations and methods of the disclosure can achieve immediate relief of the acute symptoms (e.g., pain and inflammation) of these lower back pain or conditions and additionally provide an extended or long term therapy (e.g., slowing, arresting, reversing or otherwise inhibiting structural damage to tissues associated with progressive disease), while avoiding long term systemic side effects associated with corticosteroid administration, including, for example, HPA suppression.

In some embodiments, an extended release formulation of fluticasone propionate and polymer matrix is administered locally to treat pain and/or inflammation in a small to medium synovial joint. Local administration of an FP/polymer extended-release formulation can occur, for example, by injection into intra-articular space, peri-articular space, soft tissues, or lesions, at or near the site of a patient's pain. In some embodiments, an extended release form of FP is administered (e.g., by single injection or as sequential injections) into a synovial joint cavity for the treatment of pain and/or inflammation in a small to medium synovial joint. In some embodiments, an extended release formulation of FP and a PLGA copolymer matrix is administered (e.g., by single injection or as sequential injections) into a synovial joint cavity or into soft tissues to slow, arrest, reverse or otherwise inhibit structural damage to tissues associated with pain and/or inflammation in a small to medium synovial joint. In some embodiments, an extended release formulation of FP and a PLA copolymer matrix is administered (e.g., by single injection or as sequential injections) into a synovial joint cavity or into soft tissues to slow, arrest, reverse or otherwise inhibit structural damage to tissues associated with pain and/or inflammation in a small to medium synovial joint. In some embodiments, the patient with pain and/or inflammation in a small to medium synovial joint has osteoarthritis.

The formulations and methods of the disclosure can achieve immediate relief of the acute symptoms (e.g., pain and inflammation) of these small to medium synovial joints and additionally provide an extended or long term therapy (e.g., slowing, arresting, reversing or otherwise inhibiting structural damage to tissues associated with progressive disease), while avoiding long term systemic side effects associated with corticosteroid administration, including, for example, HPA suppression. In some embodiments, the patient with pain and/or inflammation in a small to medium synovial joint has osteoarthritis.

In some embodiments, an extended release formulation of fluticasone propionate and polymer matrix is administered locally to treat pain and/or inflammation in a large synovial joint. Local administration of an FP/polymer extended-release formulation can occur, for example, by injection into intra-articular space, peri-articular space, soft tissues, or lesions, at or near the site of a patient's pain. In some embodiments, an extended release form of FP is administered (e.g., by single injection or as sequential injections) into a synovial joint cavity for the treatment of pain and/or inflammation in a large synovial joint. In some embodiments, an extended release formulation of FP and a PLGA copolymer matrix is administered (e.g., by single injection or as sequential injections) into a synovial joint cavity or into soft tissues to slow, arrest, reverse or otherwise inhibit structural damage to tissues associated with pain and/or inflammation in a large synovial joint. In some embodiments, an extended release formulation of FP and a PLA copolymer matrix is administered (e.g., by single injection or as sequential injections) into a synovial joint cavity or into soft tissues to slow, arrest, reverse or otherwise inhibit structural damage to tissues associated with pain and/or inflammation in a large synovial joint. In some embodiments, the patient with pain and/or inflammation in a large synovial joint has osteoarthritis.

The formulations and methods of the disclosure can achieve immediate relief of the acute symptoms (e.g., pain and inflammation) of these large synovial joints and additionally provide an extended or long term therapy (e.g., slowing, arresting, reversing or otherwise inhibiting structural damage to tissues associated with progressive disease), while avoiding long term systemic side effects associated with corticosteroid administration, including, for example, HPA suppression. In some embodiments, the patient with pain and/or inflammation in a large synovial joint has osteoarthritis.

In some embodiments, the size range of the particles used in the FP/polymer extended-release formulations provided herein is tailored or otherwise modified based on the desired indication that is to be treated. For example, particles having a mean diameter larger than 6-7 μm are at risk for adverse events when administered to the spine, such as, for example, arterial infarction in the spine. However, to ensure maximal release of the FP from the FP/polymer formulation, it is desirable to maximize particle size, while simultaneously constraining the particle size distribution to exclude the risk of individual particles or aggregates above 6-7 μm. Accordingly, in some embodiments for the treatment of spinal-related indications, the particles used in the one or more epidural injections in these embodiments can be in the 100 nm to 6 μm range, provided that the upper end of this range is subject to stringent particle control and distribution conditions and techniques. In some embodiments for the treatment of spinal-related indications, the particles used in the one or more epidural injections is in the range of about 100 nm to 3 μm range.

In some embodiments for the treatment of synovial joint-related indications, the particles used in the one or more injections in these embodiments can be in the 100 nm to 10 μm range, as the use of larger diameter particles can maximize joint residency time and/or can maximize drug release into the synovial joint cavity and surrounding areas, while minimizing the available surface area of the particle. In some embodiments for the treatment of synovial joint-related indications, the particles used in the one or more injections in these embodiments are in the 100 nm to 6 μm range. In some embodiments for the treatment of synovial joint-related indications, the particles used in the one or more injections in these embodiments are in the 100 nm to 3 μm range.

In some embodiments, a formulation is provided wherein a nanoparticle matrix, such as, for example, matrices that contain or are derived from PLGA, PLA, hydrogels, hyaluronic acid, etc., incorporates fluticasone propionate, and the FP extended-release formulation provides at least two weeks, preferably at least three weeks, including up to and beyond 30 days, or 60 days, or 90 days, or 120 days, or 180 days, or 210 days, or 270 days, or beyond 270 days of a sustained, steady state release of the FP.

In some embodiments, a formulation is provided wherein a small microparticle matrix, such as, for example, matrices that contain or are derived from PLGA, PLA, hydrogels, hyaluronic acid, etc., incorporates fluticasone propionate, and the FP extended-release formulation provides at least two weeks, preferably at least three weeks, including up to and beyond 30 days, or 60 days, or 90 days, or 120 days, or 180 days, or 210 days, or 270 days, or beyond 270 days of a sustained, steady state release of the FP.

The FP extended-release formulations of the disclosure retain sustained efficacy even after the FP is no longer resident at the site of administration, for example, in the epidural space, and/or after the FP is no longer detected in the systemic circulation. The FP extended-release formulation retains sustained efficacy even after the FP extended-release formulation ceases to release therapeutically effective amounts of FP. For example, in some embodiments, the FP released by the extended-release formulation retains efficacy for at least one week, at least two weeks, at least three weeks, at least four weeks, at least five weeks, at least six weeks, at least seven weeks, at least eight weeks, at least nine weeks, at least 12 weeks, at least 15 weeks, at least 18 weeks, at least 21 weeks, at least 24 weeks, or more than 24 weeks post-administration.

In some embodiments, a controlled or sustained-release formulation is provided wherein a polymer matrix (such as, for example, matrices that contain or are derived from PLGA, hydrogels, hyaluronic acid, etc.) incorporates fluticasone propionate, and the formulation may or may not exhibit an initial rapid release, also referred to herein as an initial “burst” of the FP for a first length of time of between 0 and 14 days, for example, between the beginning of day 1 through the end of day 14, in addition to the sustained, steady state release of FP for a second length of time of at least two weeks, preferably at least three weeks, including up to and beyond 30 days, or 60 days, or 90 days, or 120 days, or 180 days, or beyond 180 days. It should be noted that when FP levels are measured in vitro, an occasional initial burst of FP release from the extended-release formulation can be seen, but this initial burst may or may not be seen in vivo. In another embodiment, a controlled or sustained-release formulation is provided wherein a polymer matrix (such as, for example, matrices that contain or are derived from PLGA, hydrogels, hyaluronic acid, etc.) incorporates fluticasone propionate, and the formulation may or may not exhibit an initial rapid release, also referred to herein as an initial “burst” of the FP for a first length of time of between 0 and 14 days, e.g., between the beginning of day 1 through the end of day 14, in addition to the sustained, steady state release of the FP for a second length of time of at least two weeks, preferably at least three weeks, including up to and beyond 30 days, or 60 days, or 90 days. In some embodiments, the length of sustained release is between 21 days and 90 days. In some embodiments, the length of sustained release is between 21 days and 60 days. In some embodiments, the length of sustained release is between 14 days and 30 days. In some embodiments, the length of release of the initial “burst” component is between 0 and 10 days, for example between the beginning of day 1 through the end of day 10. In some embodiments, the length of release of the initial “burst” component is between 0 and 6 days, for example between the beginning of day 1 through the end of day 6. In some embodiments, the length of initial “burst,” component is between 0 and 2 days, for example between the beginning of day 1 through the end of day 2. In some embodiments, the length of initial “burst” component is between 0 and 1 day, for example between the beginning of day 1 through the end of day 1.

Upon administration, the FP extended-release formulation may provide an initial release of FP at the site of administration, for example, in the epidural space or into the synovial joint cavity. Once the initial release of FP has subsided, the controlled or sustained release of the FP extended-release formulations continues to provide therapeutic (e.g., epidural or synovial) concentrations of FP at or near the site of injection to suppress lower back pain and inflammation, maintain analgesia, and/or slow, arrest or reverse structural damage to tissues for an additional period of therapy following administration. In some embodiments, the length of sustained release is between 21 days and 90 days. In some embodiments, the length of sustained release is between 21 days and 60 days. In some embodiments, the length of sustained release is between 14 days and 30 days. In some embodiments, the length of release of the immediate release form is between 1 day and 14 days. In some embodiments, the length of release of the immediate release form is between 1 day and 10 days. In some embodiments, the length of release of the immediate release form is between 1 day and 8 days. In some embodiments, the length of release of the immediate release form is between 1 day and 6 days. In some embodiments, the length of release of the immediate release form is between 1 day and 4 days.

In some embodiments, the local concentration of FP at or near the injection site, e.g., the epidural and/or synovial concentration of FP, is maintained for a duration of at least 50 days. In some embodiments, the local concentration of FP at or near the injection site, e.g., the epidural and/or synovial concentration of FP, is maintained for a duration of at least 75 days. In some embodiments, the local concentration of FP at or near the injection site, e.g., the epidural and/or synovial concentration of FP, is maintained for a duration of at least 90 days. In some embodiments, the local concentration of FP at or near the injection site, e.g., the epidural and/or synovial concentration of FP, is maintained for a duration of at least 180 days. In some embodiments, the local concentration of FP at or near the injection site, e.g., the epidural and/or synovial concentration of FP, is maintained for a duration of at least four months or longer. In some embodiments, the local concentration of FP at or near the injection site, e.g., the epidural and/or synovial concentration of FP, is maintained for a duration of at least five months or longer. In some embodiments, the local concentration of FP at or near the injection site, e.g., the epidural and/or synovial concentration of FP, is maintained for a duration of at least six months or longer. In some embodiments, the local concentration of FP at or near the injection site, e.g., the epidural and/or synovial concentration of FP, is maintained for a duration of at least seven months or longer. In some embodiments, the local concentration of FP at or near the injection site, e.g., the epidural and/or synovial concentration of FP, is maintained for a duration of at least eight months or longer. In some embodiments, the local concentration of FP at or near the injection site, e.g., the epidural and/or synovial concentration of FP, is maintained for a duration of at least nine months or longer. In some embodiments, the local concentration of FP at or near the injection site, e.g., the epidural and/or synovial concentration of FP, is maintained for a duration of at least 10 months or longer. In some embodiments, the local concentration of FP at or near the injection site, e.g., the epidural and/or synovial concentration of FP, is maintained for a duration of at least 11 months or longer. In some embodiments, the local concentration of FP at or near the injection site, e.g., the epidural and/or synovial concentration of FP, is maintained for a duration of at least 12 months or longer. In some embodiments, the local concentration of FP at or near the injection site, e.g., the epidural and/or synovial concentration of FP, is maintained for a duration in the range of about 3 months to at least about 12 months.

In some embodiments, the local concentration of FP at or near the injection site, e.g., the epidural and/or synovial concentration of FP, is maintained by administering at least one additional dose of FP. In some embodiments, at least one additional dose of FP is administered as an extended-release formulation. In some embodiments, at least one additional dose of FP is administered as an extended release, e.g., a controlled- or sustained-release formulation. In some embodiments, FP is released from the formulation for a duration of at least between 14 days and 90 days. In some embodiments, FP is released from the formulation for a duration of at least between 30 days and 90 days. In some embodiments, FP is released from the formulation for a duration of at least 3 months. In some embodiments, FP is released from the formulation for a duration of at least between 3 months and 12 months. In some embodiments, FP is released from the formulation for a duration of at least between 3 months and 6 months. In some embodiments, FP is released from the formulation for a duration of at least between 6 months and 12 months.

The disclosure also provides methods for maximizing analgesic effect and maintaining maximal analgesic effect over a prolonged duration in a patient with a disease or disorder associated with pain and/or lower back inflammation by administering to a subject in need thereof an extended release FP/polymer formulation, e.g., a controlled- or sustained-release FP/polymer formulation, and maintaining a local concentration of FP at or near the site of injection of at least about 0.95 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining a local concentration of FP at or near the site of injection of at least about 0.95 ng/ml to about 157.58 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

The disclosure also provides methods for maximizing analgesic effect and maintaining maximal analgesic effect over a prolonged duration in a patient with a disease or disorder associated with lower back pain and/or lower back inflammation by administering to a subject in need thereof an extended release FP/polymer formulation, e.g., a controlled- or sustained-release FP/polymer formulation, and maintaining an epidural concentration of FP of at least about 0.95 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining an epidural concentration of FP of at least about 0.95 ng/ml to about 157.58 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

The disclosure also provides methods for maximizing analgesic effect and maintaining maximal analgesic effect over a prolonged duration in a patient with pain and/or inflammation in a small to medium synovial joint by administering to a subject in need thereof an extended release FP/polymer formulation, e.g., a controlled- or sustained-release FP/polymer formulation, and maintaining an synovial concentration of FP of at least about 0.95 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining an synovial concentration of FP of at least about 0.95 ng/ml to about 157.58 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

The disclosure also provides methods for maximizing analgesic effect and maintaining maximal analgesic effect over a prolonged duration in a patient with pain and/or inflammation in a large synovial joint by administering to a subject in need thereof an extended release FP/polymer formulation, e.g., a controlled- or sustained-release FP/polymer formulation, and maintaining an synovial concentration of FP of at least about 0.95 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months. In some embodiments, the method includes maintaining an synovial concentration of FP of at least about 0.95 ng/ml to about 157.58 ng/ml for a duration of at least 24 days, for example, at least 3 months, at least 4 months, at least 5 months, at least 6 months, at least 7 months, at least 8 months, at least 9 months, at least 10 months, at least 11 months, or at least 12 months.

### Definitions

The following terms are used throughout this disclosure.

The terms “fluticasone propionate”, “fluticasone”, “FP”, and the like are used interchangeably herein.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. Even more preferred humans would be those with type I or type II diabetes with chronic, non-healing infected wounds. A subject may also be a pet animal, including dogs, cats and horses. Preferred agricultural animals would be cattle and goats. In some embodiments, “patient” refers to a human diagnosed with a disease or condition that can be treated in accordance with the disclosure described herein. In some embodiments it is contemplated that the formulations described herein may also be used in horses and other animals.

“Delivery” refers to any means used to place the drug into a patient. Such means may include without limitation, placing matrices into a patient that release the drug into a target area. One of ordinary skill in the art recognizes that the matrices may be delivered by a wide variety of methods, e.g., injection by a syringe, placement into a drill site, catheter or cannula assembly, or forceful injection by a gun type apparatus or by placement into a surgical site in a patient during surgery.

The terms “treatment” and “treating” a patient refer to reducing, alleviating, stopping, blocking, delaying the onset of, delaying the progression of the disease state and/or the symptoms of the disease state, or preventing the symptoms of pain and/or inflammation in a patient. As used herein, “treatment” and “treating” includes partial alleviation of symptoms as well as complete alleviation of the symptoms for a time period. The time period can be hours, days, months, or even years.

By an “effective” amount or a “therapeutically effective amount” of a drug or pharmacologically active agent is meant a nontoxic but sufficient amount of the drug or agent to provide the desired effect, e.g., analgesia. An appropriate “effective” amount in any individual case may be determined by one of ordinary skill in the art using routine experimentation.

“Site of a patient's pain” refers to any area within a body causing pain, e.g., lower back pain or pain radiating from epidural or perineural spaces. The pain perceived by the patient may result from inflammatory responses, mechanical stimuli, chemical stimuli, thermal stimuli, as well as allodynia. The site of a patient's pain can comprise one or multiple sites in the spine, such as between the cervical, thoracic, or lumbar vertebrae, or can comprise one or multiple sites located within the immediate area of an inflamed or injured lower back area.

The terms “pharmaceutically acceptable” and “veterinarily acceptable” refer to a pharmaceutically- or veterinarily-acceptable material, composition, or vehicle, such as a liquid or solid filler, diluent, excipient, solvent, or encapsulating material. Each component must be “pharmaceutically acceptable” or “veterinarily acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. (See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004)).

A “biocompatible” material refers to a material that is not toxic to the human body, it is not carcinogenic and it should induce limited or no inflammation in body tissues. A “biodegradable” material refers to a material that is degraded by bodily processes (e.g., enzymatic) to products readily disposable by the body or absorbed into body tissue. The biodegraded products should also be biocompatible with the body. In the context of intra-articular drug delivery systems for fluticasone propionate and other corticosteroids, such polymers may be used to fabricate, without limitation: small microparticles, nanoparticles, micro-spheres, matrices, small microparticle matrices, nanoparticle matrices, micro-sphere matrices, capsules, hydrogels, rods, wafers, pills, liposomes, fibers, pellets, or other appropriate pharmaceutical delivery compositions that a physician can administer into the joint. The biodegradable polymers degrade into non-toxic residues that the body easily removes or break down or dissolve slowly and are cleared from the body intact. The polymers may be cured ex-vivo forming a solid matrix that incorporates the drug for controlled release to an inflammatory region. Suitable biodegradable polymers may include, without limitation natural or synthetic biocompatible biodegradable material. Natural polymers include, but are not limited to, proteins such as albumin, collagen, gelatin synthetic poly(aminoacids), and prolamines; glycosaminoglycans, such as hyaluronic acid and heparin; polysaccharides, such as alginates, chitosan, starch, and dextrans; and other naturally occurring or chemically modified biodegradable polymers. Synthetic biocompatible biodegradable materials include, but are not limited to, poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), polyurethanes, polythioesters, poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer (polyactive) (copolymer of poly(ethyleneglycol)-terepthalate and poly(butylene-terepthalate)), methacrylates, poly(N-isopropylacrylamide), PEO-PPO-PEO (pluronics) (copolymer of poly(ethylene oxide)-poly(propylene oxide)-poly(ethylene oxide)), PEO-PPO-PAA copolymers (copolymers of poly(ethylene oxide)-poly(propylene oxide)-poly(acrylic acid)), and PLGA-PEO-PLGA (copolymers of poly(lactide-co-glycolic acid)-poly(ethylene oxide)-poly(lactide-co-glycolic acid)) blends and copolymers thereof and any combinations thereof. The biocompatible biodegradable material can include a combination of biocompatible biodegradable materials. For example, the biocompatible biodegradable material can be a triblock, or other multi-block, formation where a combination of biocompatible biodegradable polymers are joined together. For example, the triblock can be PLGA-PEG-PLGA.

The terms “administering”, “administer”, “administration” and the like, as used herein, refer to any mode of transferring, delivering, introducing, or transporting any of the formulations described herein to a subject in need of treatment. Such modes include, but are not limited to, intra-articular, oral, topical, intravenous, intraperitoneal, intramuscular, intradermal, intranasal, and subcutaneous administration.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

As used herein, the expression “wherein a compound comprises between X % to Y % of the microparticles” is understood to mean that the microparticles comprise X % to Y % of the compound, by weight of the microparticles.

As used herein, all references to “mean diameter” for both small microparticles and nanoparticles refer to the “volumetric mean diameter” (as opposed to the “number mean diameter”). Volumetric mean diameter is the generally accepted metric in Pharmaceutical industry.

Unless specified otherwise, as referred to herein, any reference to a percentage is a reference to percentage by weight.

Descriptions of various embodiments of the disclosure are given below. Although these embodiments are exemplified with reference to treat joint pain associated with osteoarthritis, rheumatoid arthritis and other joint disorders, it should not be inferred that the disclosure is only for these uses. Rather, it is contemplated that embodiments of the present invention will be useful for treating other forms of joint pain by administration into articular and peri-articular spaces. In addition, it will be understood that for some embodiments injection near a joint may be equivalent to injections in that joint. Any and all uses of specific words and references are simply to detail different embodiments of the present disclosure.

Local administration of a corticosteroid formulation, e.g., an FP formulation, can occur, for example, by injection into the epidural space at or near the site of a patient's pain and/or structural tissue damage. Local injection of the formulations described herein into epidural spaces may be useful in the treatment of, for example, lower back pain and/or lower back inflammation. In some embodiments, the lower back pain and/or inflammation is chronic lower back pain and/or inflammation. In some embodiments, the lower back pain and/or inflammation is acute lower back pain and/or inflammation.

In one embodiment, the FP formulations provided herein are useful in treating, alleviating a symptom of, ameliorating and/or delaying the progression of sciatica. In one embodiment, the FP formulations provided herein are useful in treating, alleviating a symptom of, ameliorating and/or delaying the progression of one or more herniated discs, including radiculopathy due to lumbar disc herniation.

In one embodiment, the FP formulations provided herein are useful in treating, alleviating a symptom of, ameliorating and/or delaying the progression of pain and/or inflammation in a small to medium synovial joint. In some embodiments, the patient with pain and/or inflammation in a small to medium synovial joint has osteoarthritis.

In one embodiment, the FP formulations provided herein are useful in treating, alleviating a symptom of, ameliorating and/or delaying the progression of pain and/or inflammation in a large synovial joint. In some embodiments, the patient with pain and/or inflammation in a large synovial joint has osteoarthritis.

Administration of an FP formulation to a patient suffering from pain and/or inflammation, such as, for example, lower back pain and/or inflammation, pain and/or inflammation in a small to medium synovial joint, or pain and/or inflammation in a large synovial joint, is considered successful if any of a variety of laboratory or clinical results is achieved. For example, administration of an FP formulation is considered successful if one or more of the symptoms associated with the pain and/or inflammation, e.g., lower back pain and/or inflammation, pain and/or inflammation in a small to medium synovial joint, or pain and/or inflammation in a large synovial joint, is alleviated, reduced, inhibited or does not progress to a further, i.e., worse, state. Administration of an FP formulation is considered successful if the pain and/or inflammation, e.g., lower back pain and/or inflammation, pain and/or inflammation in a small to medium synovial joint, or pain and/or inflammation in a large synovial joint, or any symptom thereof enters remission or does not progress to a further, i.e., worse, state.

Successful efficacy of administration of an FP extended-release formulation is assessed using any of a variety of art-recognized methods. For example, efficacy can be assessed using weekly average of daily (24 hour) pain intensity score, and patient and clinical global impression of change. Efficacy can also be assessed by monitoring evidence of inflammation in a patient. For example, evidence of inflammation can be assessed by monitoring signs of local inflammation including tenderness, swelling, redness/heat, and/or effusion at various time intervals.

Also, any and all alterations and further modifications of the disclosure, as would occur to one of ordinary skill in the art, are intended to be within the scope of the disclosure.

Fluticasone Propionate:

Fluticasone propionate (FP) belongs to a class of drugs known as corticosteroids, specifically glucocorticoids, which are hormones that predominantly affect the metabolism of carbohydrates and, to a lesser extent, fat and protein. Corticosteroid molecules have the following basic structure:

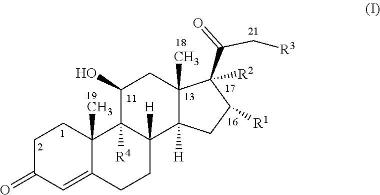

There are four classes of corticosteroids (Class A, Class B, Class C, and Class D) (See e.g., Foti et al. “Contact Allergy to Topical Corticosteroids: Update and Review on Cross-Sensitization.” Recent Patents on Inflammation & Allergy Drug Discovery 3 (2009): 33-39; Coopman et al., “Identification of cross-reaction patterns in allergic contact dermatitis to topical corticosteroids.” Br J Dermatol 121 (1989): 27-34). FP is a Class D corticosteroid, which are clobetasone or hydrocortisone esterified types with a long chain on C17 and/or C21 and with no methyl group on C16.

FP is the propionate salt form of fluticasone, a synthetic tri-fluorinated glucocorticoid receptor agonist with anti-allergic, anti-inflammatory, and anti-pruritic effects. The structure of FP is shown below:

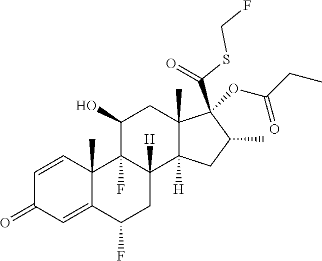

Binding and activation of the glucocorticoid receptor results in the activation of lipocortin that, in turn, inhibits cytosolic phospholipase A2, which triggers cascade of reactions involved in synthesis of inflammatory mediators, such as prostaglandins and leukotrienes. Secondly, mitogen-activated protein kinase (MAPK) phosphatase 1 is induced, which thereby leads to dephosphorylation and inactivation of Jun N-terminal kinase directly inhibiting c-Jun mediated transcription. Finally, transcriptional activity of nuclear factor (NF)-kappa-B is blocked, thereby inhibits the transcription of cyclooxygenase 2, which is essential for prostaglandin production.

For the present disclosure, non-limiting examples of fluticasone propionate include fluticasone propionate and/or pharmaceutically acceptable salts thereof.

Embodiments of the disclosure include using extended release, e.g., sustained release, FP formulations delivered to treat pain at dosages that do not exhibit adverse side effects that can be seen with long-term administration of corticosteroids, including, by way of non-limiting example, adverse suppression of the HPA axis.

Extended Release Delivery Platforms, Controlled or Sustained Release Delivery Platforms:

The disclosure encompasses any extended release fluticasone propionate (FP) formulation, e.g., any controlled- or sustained-release FP formulation, that produces and maintains maximal analgesic effect in a patient for an extended period of time post administration. Suitable formulations can vary in composition, components, dosing, etc., provided that the extended-release FP formulation is effective to deliver the FP over an extended period of time and to maintain a local concentration of FP at or near the site of injection, e.g., an epidural or synovial concentration of FP, in the desired range.

In some embodiments, the extended release formulation, e.g., the sustained release formulation, includes a matrix, such as for example, a hydrogel-based matrix, a hyaluronic acid-based matrix, and/or a biodegradable polymer-based matrix. In some embodiments, the hydrogel is a polyurethane hydrogel, a polyacrylate hydrogel, a gelatin hydrogel, a carboxymethyl cellulose hydrogel, a pectin hydrogel, an alginate hydrogel, and/or a hyaluronic acid hydrogel. In some embodiments, the biodegradable polymer is selected from, but not limited to, PLGA, PLA, PGA, polycaprolactone, polyhydroxybutyrate, polyorthoesters, polyalkaneanhydrides, gelatin, collagen, oxidized cellulose, and/or polyphosphazene.

In some embodiments, the extended release formulation, e.g., the sustained release formulation, includes a biodegradable polymer nanoparticle formulation and/or a biodegradable polymer small microparticle formulation. The manufacture of extended release nanoparticles, e.g., sustained-release nanoparticles, or methods of making biodegradable polymer nanoparticles are known in the art. The manufacture of extended release small microparticles, e.g., sustained-release small microparticles, or methods of making biodegradable polymer small microparticles are known in the art.

In some embodiments, the extended release formulation, e.g., the sustained release formulation, includes a biodegradable polymer that may include, without limitation, natural or synthetic biocompatible biodegradable materials. Natural polymers include, but are not limited to, proteins such as albumin, collagen, gelatin synthetic poly(aminoacids), and prolamines; glycosaminoglycans, such as hyaluronic acid and heparin; polysaccharides, such as alginates, chitosan, starch, and dextrans; and other naturally occurring or chemically modified biodegradable polymers. Synthetic biocompatible biodegradable materials include, but are not limited to the group comprising of, poly(lactide-co-glycolide) (PLGA), polylactide (PLA), polyglycolide (PG), polyhydroxybutyric acid, poly(trimethylene carbonate), polycaprolactone (PCL), polyvalerolactone, poly(alpha-hydroxy acids), poly(lactones), poly(amino-acids), poly(anhydrides), polyketals poly(arylates), poly(orthoesters), poly(orthocarbonates), poly(phosphoesters), poly(ester-co-amide), poly(lactide-co-urethane, polyethylene glycol (PEG), polyvinyl alcohol (PVA), PVA-g-PLGA, PEGT-PBT copolymer(polyactive), polyurethanes, polythioesters, methacrylates, poly(N-isopropylacrylamide), PEO-PPO-PEO (pluronics), PEO-PPO-PAA copolymers, and PLGA-PEO-PLGA blends and copolymers thereof, multi-block polymer configurations such as PLGA-PEG-PLGA, and any combinations thereof. These polymers may be used in making controlled release or sustained release compositions disclosed herein.

In some embodiments, the extended release formulation, e.g., the sustained-release nanoparticle, the sustained release small microparticle, or other sustained-release formulation, is poly(d,l-lactic-co-glycolic acid) (PLGA) or is PLGA-based. PLGA polymers are commercially available from a number of sources. If not purchased from a supplier, then the biodegradable PLGA copolymers may be prepared by the procedure set forth in U.S. Pat. No. 4,293,539 (Ludwig, et al.), the disclosure of which is hereby incorporated by reference in its entirety. Ludwig prepares such copolymers by condensation of lactic acid and glycolic acid in the presence of a readily removable polymerization catalyst (e.g., a strong acid ion-exchange resin such as Dowex HCR-W2-H). However, any suitable method known in the art of making the polymer can be used.

In some embodiments, the extended release formulation, e.g., the sustained-release nanoparticle, the sustained release small microparticle, or other sustained-release formulation, is polylactic acid (PLA) or is PLA-based. PLA polymers are commercially available from a number of sources. If not purchased from a supplier, then the biodegradable PLA copolymers can be prepared using any suitable method known in the art for making PLA polymers.

Any suitable polymers known in the art can be used in the FP extended- or sustained-release formulations described herein. By way of non-limiting example, PLGA, PLA, PLGA-PEG, PLA-PEG can be used. Suitable molecular weight ranges of PLGA or PLA can be between 1 and 80 kDa (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80). Likewise, the molecular weight of the PEG can range from 1-8 kDa (i.e., 1, 2, 3, 4, 5, 6, 7, and 8).

In any of the formulations described herein, the polymer can comprise an end cap (e.g., an ester end cap (“E”) and/or an acid end cap (“A”)). Those skilled in the art will recognize that the nature of the end cap selected (i.e., ester vs. acid), alone or in combination with the molecular weight of the polymer, can play a role in the duration of the FP formulations described herein.

Those skilled in the art will recognize that polymers can be defined by a combination of their average molecular weight range and their inherent viscosity. Selecting the appropriate polymer will influence the in vitro release kinetics of the resulting FP formulations.

PLGA- or PLA-based small microparticles and/or nanoparticles can be made by, but not limited to, single or double emulsion method, spray drying, solvent evaporation, phase separation, fluidized bed coating or combinations thereof.

In the coacervation process, a suitable biodegradable polymer is dissolved in an organic solvent. Suitable organic solvents for the polymeric materials include, but are not limited to acetone, halogenated hydrocarbons such as chloroform and methylene chloride, aromatic hydrocarbons such as toluene, halogenated aromatic hydrocarbons such as chlorobenzene, and cyclic ethers such as dioxane. The organic solvent containing a suitable biodegradable polymer is then mixed with a non-solvent or anti-solvent such as silicone based solvent. By mixing the miscible non-solvent in the organic solvent, the polymer precipitates out of solution in the form of liquid droplets. The liquid droplets are then mixed with another non-solvent, such as heptane or petroleum ether, to form the hardened small microparticles and/or hardened nanoparticles. The small microparticles and/or nanoparticles are then collected and dried. Process parameters such as solvent and non-solvent selections, polymer/solvent ratio, temperatures, stirring speed and drying cycles are adjusted to achieve the desired particle size, surface smoothness, and narrow particle size distribution.

Phase separation or phase inversion procedures entrap dispersed agents in the polymer to prepare small microparticles and/or nanoparticles. Phase separation is similar to coacervation of a biodegradable polymer. By addition of a non-solvent such as petroleum ether, to the organic solvent containing a suitable biodegradable polymer, the polymer is precipitated from the organic solvent to form small microparticles and/or nanoparticles.

In the salting out process, a suitable biodegradable polymer is dissolved in an aqueous miscible organic solvent. Suitable water miscible organic solvents for the polymeric materials include, but are not limited to acetone, acetonitrile, and tetrahydrofuran. The water miscible organic solvent containing a suitable biodegradable polymer is then mixed with an aqueous solution containing salt. Suitable salts include, but are not limited to electrolytes such as magnesium chloride, calcium chloride, or magnesium acetate and non-electrolytes such as sucrose. The polymer precipitates from the organic solvent to form small microparticles and/or nanoparticles, which are collected and dried. Process parameters such as solvent and salt selection, polymer/solvent ratio, temperatures, stirring speed and drying cycles are adjusted to achieve the desired particle size, surface smoothness, and narrow particle size distribution.

Alternatively, the small microparticles and/or nanoparticles may be prepared by the process of Ramstack et al., 1995, described in published international patent application WO 95/13799, the disclosure of which is incorporated herein in its entirety. The Ramstack et al. process essentially provides for a first phase, including an active agent and a polymer, and a second phase, that are pumped through a static mixer into a quench liquid to form small microparticles and/or nanoparticles containing the active agent. The first and second phases can optionally be substantially immiscible and the second phase is preferably free from solvents for the polymer and the active agent and includes an aqueous solution of an emulsifier.

In the spray drying process, a suitable biodegradable polymer is dissolved in a suitable solvent and then sprayed through nozzles into a drying environment provided with sufficient elevated temperature and/or flowing air to effectively extract the solvent.

Alternatively, a suitable biodegradable polymer can be dissolved or dispersed in supercritical fluid, such as carbon dioxide. The polymer is either dissolved in a suitable organic solvent, such as methylene chloride, prior to mixing in a suitable supercritical fluid or directly mixed in the supercritical fluid and then sprayed through a nozzle. Process parameters such as spray rate, nozzle diameter, polymer/solvent ratio, and temperatures, are adjusted to achieve the desired particle size, surface smoothness, and narrow particle size distribution.

In a fluidized bed coating, the drug is dissolved in an organic solvent along with the polymer. The solution is then processed, e.g., through a Wurster air suspension coating apparatus to form the final microcapsule product. In some embodiments, the microcapsule product is formed using a spinning disk methodology, e.g., a Southwest Research Institute (SwRI) technology.

The small microparticles and/or nanoparticles can be prepared in a size distribution range suitable for local infiltration or injection. The diameter and shape of the small microparticles and/or nanoparticles can be manipulated to modify the release characteristics. In addition, other particle shapes, such as, for example, cylindrical shapes, can also modify release rates of an extended release FP formulation, e.g., a sustained release FP formulation, by virtue of the increased ratio of surface area to mass inherent to such alternative geometrical shapes, relative to a spherical shape. The formulation includes a combination of small microparticles and nanoparticles, wherein the combination of small microparticles and nanoparticles has a volumetric mean diameter ranging between about 0.1 to 10 microns. In some embodiments, the nanoparticles have a volumetric mean diameter of between about 1 microns to about 10 microns. In some embodiments, the nanoparticles have a volumetric mean diameter of between about 100 to about 1,000 microns.

Biodegradable polymer nanoparticles that deliver extended release FP formulations, e.g., sustained release FP formulations, may be suspended in suitable aqueous or non-aqueous carriers which may include, but are not limited to water, saline, pharmaceutically acceptable oils, low melting waxes, fats, lipids, liposomes and any other pharmaceutically acceptable substance that is lipophilic, substantially insoluble in water, and is biodegradable and/or eliminable by natural processes of a patient's body. Oils of plants such as vegetables and seeds are included. Examples include oils made from corn, sesame, canola, soybean, castor, peanut, olive, arachis, maize, almond, flax, safflower, sunflower, rape, coconut, palm, babassu, and cottonseed oil; waxes such as carnoba wax, beeswax, and tallow; fats such as triglycerides, lipids such as fatty acids and esters, and liposomes such as red cell ghosts and phospholipid layers.

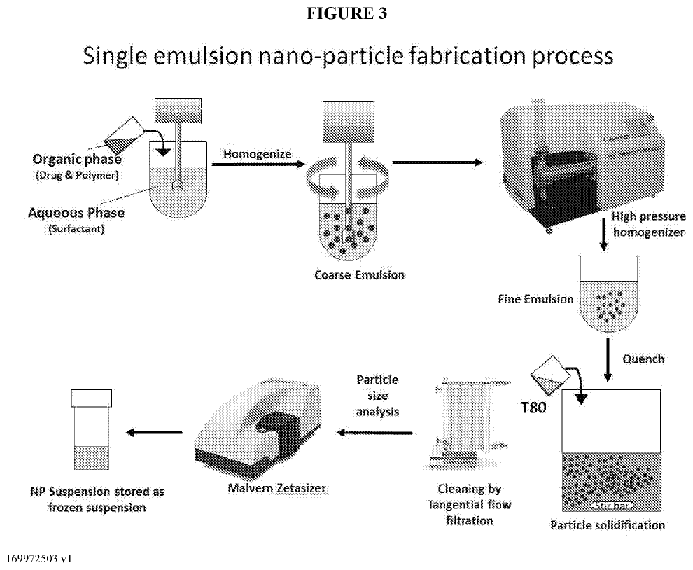

In some non-limiting embodiments, the controlled- or sustained-release FP nanoparticles are prepared using a single emulsion nano-particle fabrication process. (See FIG. 3).

Briefly, the drug and polymer are dissolved in an organic phase (i.e., a mixture of benzyl alcohol and ethyl acetate). The ratio of benzyl alcohol and ethyl acetate can vary from 100:0 to 0:100. Next, the organic phase is added to aqueous phase over homogenization which results into oil/water (o/w) emulsion. In various embodiments, the ratio of organic phase to aqueous can range from 1:2 to 1:8.

The “course” emulsion is then fed through a high pressure homogenizer (single or multiple passes) at pressure ranging anywhere from 5k to 30k PSI. Next, the fine emulsion, which is obtained by passing through the high pressure microfluidics homogenizer, is quenched into deionized (DI) water or buffered solution to solidify the particles. Finally, the nanoparticles are then cleaning using a Tangential Flow filtration system. After confirming the particle size of the nanoparticles, they are stored as a frozen suspension.

Non-limiting examples of suitable FP nano-formulations prepared by a single emulsion nano-particle fabrication process are provided in the table below:

| | Actual | | |
|---|---|---|---|
| | drug | Particle size | |
| Batch # | load | (Z-average) | Polymer Type |
| NB-0006-002-02 | 5.26% | 223.8 nm | 50-5 (PLGA-PEG) |
| | | | (copolymer of 50 kDa |
| | | | poly(lactic-co-glycolic acid) |
| | | | (PLGA) and 5 kDa |
| | | | polyethylene glycol (PEG)) |
| NB-0006-003-02 | 6.11% | 183.4 nm | 80-5 (PLGA-PEG) |
| | | | (copolymer of 80 kDa PLGA |
| | | | and 5 kDa PEG) |
| NB-0006-005-01 | 5.68% | 182.7 nm | 80-5 (PLA-PEG) |
| | | | (copolymer of 80 kDa |
| | | | poly(lactic acid) (PLA) and 5 |
| | | | kDa PEG) |
| NB-0006-005-02 | 4.19% | 108.3 nm | 80-5 (PLA-PEG) |
| | | | (copolymer of 80 kDa PLA |
| | | | and 5 kDa PEG) |

In this table, Z average is the mean particle size measured by Dynamic light scattering method which is an intensity based method.

Non-limiting examples of suitable FP nano-formulations prepared by a spray-drying method are provided in the table below:

| | Drug | | Polymer |
|---|---|---|---|
| Formulation | Load | Solvent | Concentration |
| PLGA (75:25 8E) | 15% | Dichloromethane | 2-3% |
| (poly(lactide-co-glycolic | | (DCM) | |
| acid; lactide to glycolide | | | |
| ratio = 75:25, inherent | | | |
| viscosity of polymer = | | | |
| 8; ester end cap) | | | |
| PLA 5A | 15% | Dichloromethane | 2-3% |
| (poly(lactic acid); | | (DCM) | |
| inherent viscosity = 5; | | | |
| acid end cap) | | | |

### Excipients:

The release rate of a corticosteroid, e.g., fluticasone propionate (FP), from a formulation can be modulated or stabilized by adding one or more pharmaceutically acceptable excipient(s) to the formulation. In some embodiments, additional excipient(s) may include any useful ingredient added to the biodegradable polymer depot that is not a corticosteroid or a biodegradable polymer. In some embodiments, additional excipient(s) may include a mixture of multiple polymers added to the biodegradable polymer depot to adjust the release profile as necessary. Pharmaceutically acceptable excipients may include without limitation lactose, dextrose, sucrose, sorbitol, mannitol, starches, gum acacia, calcium phosphate, alginates, tragacanth, gelatin, calcium silicate, microcrystalline cellulose, PEG, polysorbate 20, polysorbate 80, polyvinylpyrrolidone, cellulose, water, saline, syrup, methyl cellulose, and carboxymethyl cellulose. An excipient for modulating the release rate of corticosteroid, e.g., FP, from the formulation may also include without limitation pore formers, pH modifiers, solubility enhancers, reducing agents, antioxidants, and free radical scavengers.

Delivery of Corticosteroid formulations:

Parenteral administration of FP formulations of the disclosure can be effected by intra-articular injection or other injection using a needle. To inject the FP formulations into a joint, needles having a gauge of about 14-28 gauge are suitable. It will be appreciated by those skilled in the art that FP formulations of the present disclosure may be delivered to a treatment site by other conventional methods, including catheters, infusion pumps, pens devices, injection guns and the like.

### Indications

Descriptions of various embodiments of the disclosure are given below. Although these embodiments are exemplified with reference to treating lower back pain and/or inflammation, it should not be inferred that the disclosure is only for these uses. Rather, it is contemplated that embodiments of the present disclosure will be useful for treating other forms of back pain by administration into epidural spaces. In addition, it will be understood that for some embodiments injection near an epidural space may be equivalent to injections in that space. It is also contemplated that embodiments of the present disclosure may be useful for injection or administration into soft tissues or lesions. Any and all uses of specific words and references are simply to detail different embodiments of the present disclosure.

Local administration of a fluticasone propionate extended-release formulation can occur, for example, by injection into the epidural space at or near the site of a patient's pain and/or structural tissue damage. Local injection of the formulations described herein into epidural spaces may be useful in the treatment of, for example, lower back pain and/or inflammation, including, for example, radiculopathy due to lumbar disc herniation.

In one embodiment, the FP extended-release formulations provided herein are useful in treating, alleviating a symptom of, ameliorating and/or delaying the progression of sciatica.

Local administration of a fluticasone propionate extended-release formulation can occur, for example, by injection into an intra-articular space at or near the site of a patient's pain and/or structural tissue damage. Local injection of the formulations described herein into synovial joint cavities may be useful in the treatment of, for example, pain and/or inflammation in a small to medium synovial joint, such as for example, one or more interphalangeal joints, one or more metacarpophalangeal joints, one or more an elbow joints, one or more wrist joints, one or more ankle joints, and any combination thereof. Local injection of the formulations described herein into synovial joint cavities may be useful in the treatment of, for example, pain and/or inflammation in a large synovial joint, such as for example, one or more knee joints, one or more shoulder joints, one or more hip joints, and any combination thereof.

In some embodiments, the patient has pain and/or inflammation related to another indication or disease associated with pain and/or inflammation. In some embodiments, the patient has carpel tunnel syndrome. In some embodiments, the patient is suffering one or more side effects from a trigger point injection. In some embodiments, the patient has rheumatoid arthritis. In some embodiments, the patient has cervical radiculopathy. In some embodiments, the patient is suffering one or more side effects from a joint replacement, such as, for example, a knee or hip replacement. In some embodiments, the patient has Complex Regional Pain Syndromes (CRPS). In some embodiments, the patient has lumbar spinal stenosis. In some embodiments, the patient has an acute spinal injury. In some embodiments, the patient has discogenic pain.

In some embodiments, the patient has pain and/or inflammation caused by or otherwise associated with an ophthalmic indication. In some embodiments, the pain and/or inflammation caused by or otherwise associated with an ophthalmic indication is selected from the group consisting of endophthalmitis (e.g., the endogenous form and the exogenous form), macular edema (e.g., macular edema that occurs as a result of age-related macular degeneration, cataract surgery, diabetes, drug toxicity, eye injury, retinal vein occlusion (e.g., central retinal vein occlusion (CRVO) and branch retinal vein occlusion), or other inflammatory eye diseases, e.g., pseudophakic macular edema), conjunctivitis, diabetic retinopathy, dry eye, episcleritis, keratitis, optic neuritis, orbital inflammation, orbital pseudotumor, post-operative inflammation, proliferative vitreoretinopathy, retinal vasculitis, scleritis, and uveitis (e.g., (i) uveitis associated with sepsis (e.g., LPS-induced uveitis); (ii) autoimmune uveitis (e.g., uveitis associated with lupus); or (iii) uveitis associated with type II, type III, type IV, or type V hypersensitivity reactions).

In one embodiment, the FP extended-release formulations provided herein are useful in treating, alleviating a symptom of, ameliorating and/or delaying the progression of Neurogenic Claudication (NC) secondary to lumbar spinal stenosis (LSS). LSS implies spinal canal narrowing with possible subsequent neural compression (classified by anatomy or etiology). Neurogenic Claudication (NC) is a hallmark symptom of lumbar stenosis, in which the column of the spinal cord (or the canals that protect the nerve roots) narrows at the lower back. This narrowing can also occur in the spaces between the vertebrae where the nerves leave the spine to travel to other parts of the body.

The nanoparticles of the disclosure are used to treat, alleviate a symptom of, ameliorate and/or delay the progression in patients suffering from NC secondary to LSS. The FP extended-release formulations can be administered, for example, by epidural steroid injection (ESI).

Administration of a FP extended-release formulation to a patient suffering from pain and/or inflammation, such as e.g., lower back pain and/or inflammation, pain and/or inflammation in a small to medium synovial joint, or pain and/or inflammation in a large synovial joint, is considered successful if any of a variety of laboratory or clinical results is achieved. For example, administration of an FP extended-release formulation is considered successful if one or more of the symptoms associated with the pain and/or inflammation, e.g., lower back pain and/or inflammation, pain and/or inflammation in a small to medium synovial joint, or pain and/or inflammation in a large synovial joint, is alleviated, reduced, inhibited or does not progress to a further, i.e., worse, state. Administration of an FP extended-release formulation is considered successful if the pain and/or inflammation e.g., lower back pain and/or inflammation, pain and/or inflammation in a small to medium synovial joint, or pain and/or inflammation in a large synovial joint, enters remission or does not progress to a further, i.e., worse, state.

Also, any and all alterations and further modifications of the disclosure, as would occur to one of ordinary skill in the art, are intended to be within the scope of the disclosure.

All references, patents, patent applications or other documents cited are hereby incorporated by reference.

### Examples

The present disclosure is further defined in the following Examples. It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various uses and conditions.

### Example 1: Extended-Release Fluticasone Propionate Nanoparticle Formulations and/or Small Microparticle Formulations

Fluticasone propionate PLGA formulations were produced as described herein.

The following polymers were used: PLGA (75:25 DLG 8E) or PLA 5A. The extended-release FP/PLGA formulations were manufactured using a Procept spray dryer technique. The drug load was 15%, as the drug has to be completely soluble. The solvent used was DCM. The polymer concentration was in the range of about 2%-3%.

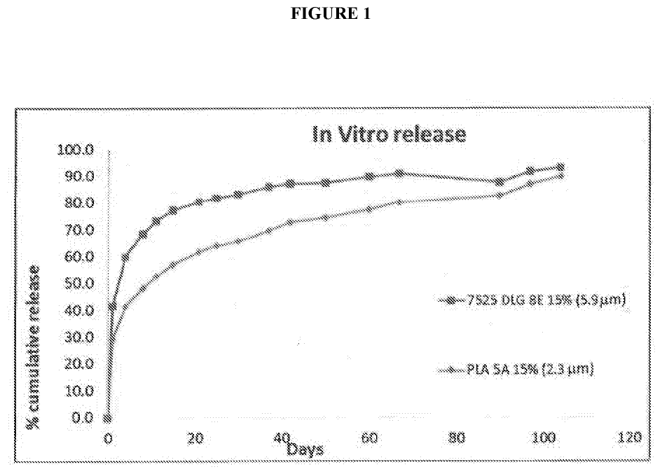

The in vitro release profile of these extended-release FP/PLGA formulations is shown in FIG. 1. This profile was produced using a release media that includes 7% SDS (sodium dodecyl sulfate) in 25 mM sodium phosphate dibasic, and a water bath at 37° C. Briefly, a fixed amount of release media is taken in small jars and put into shaking water bath which is set at 37° C. Once the media has reached 37° C., certain amount of dried microspheres are added to release media such that a “sink condition” is maintained for active pharmaceutical ingredient (API). Aliquots from supernatant are taken at fixed time points and evaluated for released drug.

In other experiments, the following polymers, which were prepared using a single emulsion nano-particle fabrication process, were used: 50k-5k PLGA-PEG (223.8 nm, 5%) (i.e., 50 kDa PLGA—5 kDa PEG; 223.8 nm mean diameter; 5% drug load); 80k-5k PLGA-PEG (183.4 nm, 7%) (i.e., 80 kDa PLGA—5 kDa PEG; 183.4 nm mean diameter; 7% drug load); 80k-5k PLA-PEG (182.7 nm, 6%) (i.e., 80 kDa PLA—5 kDa PEG; 182.7 nm mean diameter; 6% drug load); and 80k-5k PLA-PEG (108.3 nm, 4%) (i.e., 80 kDa PLA—5 kDa PEG; 108.3 mean diameter; 4% drug load).

Any other suitable polymers known in the art can be used in these formulations. By way of non-limiting example, PLGA, PLA, PLGA-PEG, PLA-PEG can be used. Suitable molecular weight ranges of PLGA or PLA can be between 1 and 80 kDa (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80). Likewise, the molecular weight of the PEG can range from 1-8 kDa (i.e., 1, 2, 3, 4, 5, 6, 7, and 8).

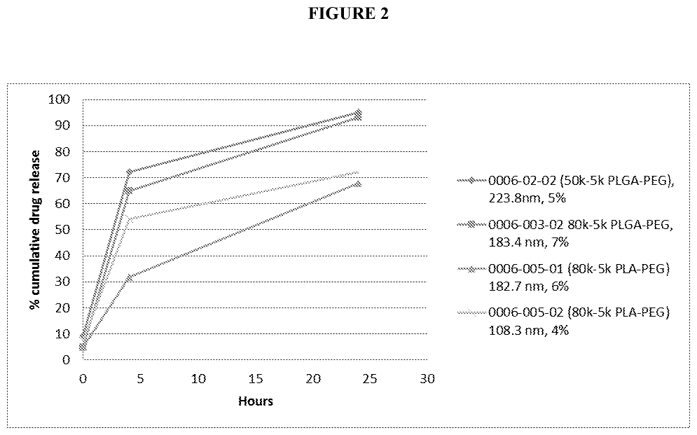

The in vitro release profile of these nano-formulations is shown in FIG. 2. This profile was produced using a release media that includes 7% SDS (sodium dodecyl sulfate) in 25 mM sodium phosphate dibasic, and a water bath at 37° C. Briefly, a fixed amount of release media is taken in small jars and put into shaking water bath which is set at 37° C. Once the media has reached 37° C., certain amount of particles (suspension or dried particles) are added to release media such that a “sink condition” is maintained for API. Aliquots from supernatant are taken at fixed time points and evaluated for released drug.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the disclosure without departing from the spirit and scope of the disclosure as defined by the claims. Other aspects, advantages, and modifications are considered to be within the scope of the following claims. The claims presented are representative of the disclosure provided herein. Other, unclaimed inventions are also contemplated. Applicants reserve the right to pursue such inventions in later claims.

What is claimed is:

1. An extended-release formulation comprising nanoparticles, small microparticles, or a combination thereof, wherein the small microparticles and/or nanoparticles comprise fluticasone propionate (FP) or a commercially available chemical analogue or a pharmaceutically-acceptable salt thereof and a polymer matrix.

2. The extended-release formulation of claim 1, wherein the polymer matrix comprises a poly(lactic-co-glycolic) acid copolymer (PLGA) copolymer matrix.

3. The extended-release formulation of claim 1, wherein the polymer matrix comprises a polylactic acid (PLA) polymer matrix.

4. The extended-release formulation of claim 1, wherein the small microparticles have a mean diameter of between 1 μm to 10 μm.

5. The extended-release formulation of claim 1, wherein the nanoparticles have a mean diameter of between 100 nm to 1000 nm.

6. The extended-release formulation of claim 1, wherein the FP is released for at least greater than 14 days.

7. The extended-release formulation of claim 1, wherein the FP is released for between 14 days and 90 days.

8. The extended-release formulation of claim 1, wherein the FP is released for between 14 days and 180 days.

9. The extended-release formulation of claim 1, wherein the extended release formulation is a controlled- or sustained-release formulation.

10. A method of treating pain or inflammation in a patient comprising administering to said patient a therapeutically effective amount of the extended-release formulation of claim 1.

11. The method of claim 10, further comprising the step of maintaining a local concentration of the FP at or near the site of administration in the range of about 0.95 ng/ml to about 157.58 ng/ml.

12. The method of claim 11, wherein the local concentration of the FP is maintained for a duration of at least 90 days.

13. The method of claim 11, wherein the local concentration of the FP is maintained for a duration of at least 180 days.

14. The method of claim 11, wherein the local concentration of the FP is maintained for a duration of at least 12 months.

15. The method of claim 11, wherein the local concentration of FP is maintained by administering at least one additional dose of FP.

16. The method of claim 15, wherein the at least one additional dose of FP is administered as an extended release formulation.

17. The method of claim 10, wherein the FP is released from the formulation for a duration of at least between 3 months and 12 months.

18. The method of claim 10, wherein the extended-release formulation is administered as one or more injections.

19. The method of claim 18, wherein the injection is one or more local injections at a site of pain.

20. The method of claim 10, wherein the pain and/or inflammation is back pain and/or inflammation, synovial joint pain and/or inflammation, ophthalmic pain and/or inflammation, a side effect of an injection in the subject, a side effect of surgical intervention in the subject, an acute injury or a side effect thereof, a chronic injury or a side effect thereof, a chronic disorder, or combinations thereof.

21. The method of claim 10, wherein the pain and/or inflammation is carpel tunnel syndrome, a trigger point injection, rheumatoid arthritis, cervical radiculopathy, knee replacement pain, hip replacement pain, Complex Regional Pain Syndromes (CRPS), lumbar spinal stenosis, and discogenic pain.

22. A method of treating lower back pain or inflammation in a patient comprising administering to said patient a therapeutically effective amount of the extended-release formulation of claim 1.

23. The method of claim 22, further comprising the step of maintaining an epidural concentration of the FP in the range of about 0.95 ng/ml to about 157.58 ng/ml.

24. The method of claim 23, wherein the epidural concentration of the FP is maintained for a duration of at least 90 days.

25. The method of claim 23, wherein the epidural concentration of the FP is maintained for a duration of at least 180 days.

26. The method of claim 23, wherein the epidural concentration of the FP is maintained for a duration of at least 12 months.

27. The method of claim 23, wherein the epidural concentration of FP is maintained by administering at least one additional dose of FP.

28. The method of claim 27, wherein the at least one additional dose of FP is administered as an extended release formulation.

29. The method of claim 22, wherein the FP is released from the formulation for a duration of at least between 3 months and 12 months.

30. The method of claim 22, wherein the extended-release formulation is administered as one or more injections.

31. The method of claim 30, wherein the injection is one or more local injections at a site of pain.

32. The method of claim 30, wherein the injection is one or more epidural injections.

33. The method of claim 22, wherein the patient has chronic lower back pain and/or inflammation.

34. The method of claim 22, wherein the patient has acute lower back pain and/or inflammation.

35. A method of treating pain and/or inflammation in a synovial joint in a patient comprising administering to said patient a therapeutically effective amount of the extended-release formulation of claim 1.

36. The method of claim 35, further comprising the step of maintaining a synovial concentration of the FP in the range of about 0.95 ng/ml to about 157.58 ng/ml.

37. The method of claim 36, wherein the synovial concentration of the FP is maintained for a duration of at least 90 days.

38. The method of claim 36, wherein the synovial concentration of the FP is maintained for a duration of at least 180 days.

39. The method of claim 36, wherein the synovial concentration of the FP is maintained for a duration of at least 12 months.

40. The method of claim 36, wherein the synovial concentration of FP is maintained by administering at least one additional dose of FP.

41. The method of claim 40, wherein the at least one additional dose of FP is administered as an extended release formulation.

42. The method of claim 35, wherein the FP is released from the formulation for a duration of at least between 3 months and 12 months.

43. The method of claim 35, wherein the extended-release formulation is administered as one or more injections.

44. The method of claim 43, wherein the injection is one or more local injections at a site of pain.

45. The method of claim 43, wherein the injection is one or more synovial injections.

46. The method of claim 35, wherein the synovial joint is a small to medium synovial joint.

47. The method of claim 46, wherein the small to medium synovial joint is one or more interphalangeal joints, one or more metacarpophalangeal joints, one or more elbow joints, one or more wrist joints, one or more ankle joints, and any combination thereof.

48. The method of claim 35, wherein the synovial joint is a large synovial joint.

49. The method of claim 48, wherein the large synovial joint is one or more knee joints.

50. The method of claim 48, wherein the patient has osteoarthritis.
